(19) United States
(12) Patent Application Publication
(10) Pub. No.: US 2023/0263169 A1
(43) Pub. Date: Aug. 24, 2023

(54) COMPOSITIONS AND METHODS FOR SCREENING PESTICIDES

(71) Applicant: Arizona Board of Regents on Behalf of the University of Arizona, Tucson, AZ (US)

(72) Inventors: Bruce E. Tabashnik, Tucson, AZ (US); Yves Carriere, Tucson, AZ (US)

(21) Appl. No.: 18/035,134

(22) PCT Filed: Nov. 3, 2021
(86) PCT No.: PCT/US2021/057809

§ 371 Date: May 3, 2023

Related U.S. Application Data
(60) Provisional application No. 63/109,364, filed on Nov. 4, 2020.

(51) Int. Cl. A01N 63/14; A01N 37/46; A01P 7/04

(52) U.S. Cl. CPC A01N 63/14; A01N 37/46; A01P 7/04

(57) ABSTRACT

Provided herein are compositions and methods for screening pesticides. In particular, provided herein are Bt resistant corn earworms, methods of generating such earworms, and the use of such earworms in screening applications.

### CROSS-REFERENCE TO RELATED APPLICATION

The present application claims priority to U.S. Provisional Patent Application No. 63/109,364, filed Nov. 4, 2020, which is hereby incorporated by reference in its entirety.

### FIELD

Provided herein are compositions and methods for screening pesticides. In particular, provided herein are Bt resistant corn earworms, methods of generating such earworms, and the use of such earworms in screening applications.

### BACKGROUND

A rapidly expanding global population, coupled with limited agricultural land, is driving farmers and industry to develop sustainable and productive methods for feeding an estimated 9 billion people by 2050. Farmers are turning to biopesticides to prevent pest damage in a more ecologically friendly manner that includes more targeted applications, lower residues and fewer applications. Benefits also include reduced environmental damage, targeted action, and reduced risk to human health.

However, there continues to be a need for new pesticides as pests evolve resistance to existing pesticides.

### SUMMARY

The present disclosure provides a genetically diverse strain of corn earworm that has been selected over many generations to be resistant to Vip3Aa, a Bt protein produced by transgenic crops. The compositions described herein find use in research, screening, and industrial applications (e.g., for screening/selecting candidate pesticides to determine if they overcome resistance).

For example, in some embodiments, provided herein is a composition comprising a variant Helicoverpa zea, wherein said Helicoverpa zea is resistant to Vip3Aa. The concentration of an insecticide killing 50% of insects tested is the LC₅₀. In some embodiments, the variant Helicoverpa zea exhibits an LC₅₀ of Vip3Aa at least 5, 10, 15, 18, 20, 30, 40, 50, 60, 70, 80, 88, 100, or 200 times higher than for wildtype Helicoverpa zea. In some embodiments, the variant Helicoverpa zea has one or more nucleic acid variations (e.g., mutations) related to resistance to Vip3 relative to wild type Helicoverpa zea.

Additional embodiments provide a kit or system, comprising any of the compositions described herein. In some embodiments, the kit or system further comprises a Vip3Aa polypeptide and/or a candidate agent. In some embodiments, the candidate agent is an insecticide (e.g., a biopesticide).

Yet other embodiments provide a method of testing a candidate agent, comprising: a) contacting a composition described herein with the candidate agent; and b) assaying the effect (e.g., effect on viability (e.g., LC₅₀ and/or growth) of the candidate agent on the Helicoverpa zea that are resistant to Vip3Aa. The present disclosure is not limited to particular candidate agents. Examples include but are not limited a pesticide (e.g., insecticide) or a biopesticide (e.g., polypeptide).

Additional embodiments are described herein.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, the term “pesticide” refers to substances that are meant to control pests. The term pesticide includes, but is not limited to, herbicides, insecticides (which may include insect growth regulators, termiticides, etc.) nematicides, molluscicides, piscicides, avicides, rodenticides, bactericides, insect repellents, animal repellents, antimicrobials, and fungicides. Most pesticides are intended to serve as plant protection products (also known as crop protection products), which in general, protect plants from weeds, fungi, or insects.

In general, a pesticide is a chemical (such as carbamate) or biological agent (such as a virus, bacterium, or fungus) that deters, incapacitates, kills, or otherwise discourages pests. Target pests can include insects, plant pathogens, weeds, molluscs, birds, mammals, fish, nematodes (roundworms), and microbes that destroy property, cause nuisance, or spread disease, or are disease vectors.

As used herein, the term “insecticide” refers to substances used to kill insects. They include ovicides and larvicides used against insect eggs and larvae, respectively. Insecticides are used in agriculture, medicine, industry and by consumers. Insecticides are classified into two major groups: systemic insecticides, which have residual or long term activity; and contact insecticides, which have no residual activity. Insecticides may be repellent or non-repellent.

In some embodiments, pesticides and/or insecticides are “biopesticides.” Biopesticides are pesticides derived from natural materials such as animals, plants, bacteria, and certain minerals. In some embodiments, biopesticides are beneficial microorganisms and/or polypeptides with insecticidal activity.

### DETAILED DESCRIPTION

Biopesticides include naturally occurring substances that control pests (e.g., biochemical pesticides), microorganisms that control pests (e.g., microbial pesticides), and pesticidal substances produced by plants containing added genetic material (plant-incorporated protectants).

Biopesticides are obtained from organisms including plants, bacteria and other microbes, fungi, nematodes, etc. They are often important components of integrated pest management (IPM) programs, and have received much practical attention as substitutes to synthetic chemical plant protection products (PPPs).

Bacillus thuringiensis, a bacterium capable of killing Lepidoptera, Coleoptera and Diptera, is a well-known insecticide example. The genes encoding some toxins from B. thuringiensis (Bt toxin) have been incorporated directly into plants through the use of genetic engineering.

Bacillus thuringiensis (or Bt) is a Gram-positive, soil-dwelling bacterium, commonly used as a biological pesticide. B. thuringiensis also occurs naturally in the gut of caterpillars of various types of moths and butterflies, as well on leaf surfaces, aquatic environments, animal feces, insect-rich environments, and flour mills and grain-storage facilities.

During sporulation, many Bt strains produce crystalline proteins (proteinaceous inclusions), called δ-endotoxins, that have insecticidal action. This has led to their use as insecticides, and more recently to genetically modified crops using Bt genes, such as Bt corn.

In 1996 another class of insecticidal proteins in Bt was discovered: the vegetative insecticidal proteins. Vip proteins do not share sequence homology with Cry proteins, in general do not compete for the same receptors, and some kill different insects than do Cry proteins. In particular, Vip3A (Estruch et al., PNAS 9:5389 (1996) is useful against a variety of pests.

However, pests can evolve resistance to biopesticides, necessitating the development of new biopesticides or modified version of existing biopesticides. Accordingly, provided herein is a Vip3Aa resistant corn earworm (H. zea) that finds use in research, screening, and industrial applications.

Helicoverpa zea, commonly known as the corn earworm, is a species (formerly in the genus Heliothis) in the family Noctuidae. The larva of the moth Helicoverpa zea is a major agricultural pest. Since it is polyphagous (feeds on many different plants) during the larval stage, the species has been given many different common names, including the cotton bollworm and the tomato fruitworm. It also consumes a wide variety of other crops.

The Vip3Aa resistant H. zea described herein exhibit increased LC₅₀ of Vip3Aa relative to wild type H. zea (e.g., at least 5, 10, 15, 18, 20, 30, 40, 50, 60, 70, 80, 88, 100, or 200 or more times wildtype H. zea). In some embodiments, Vip3Aa resistant H. zea are generated using a selection process on selective medium (e.g., comprising Vip3Aa). In some embodiments, Vip3A resistant H. zea are engineered by mutating genes involved in Vip3A resistance (e.g., identified using the methods described in Example 2).

The Vip3Aa resistant H. zea described herein find use in a variety of applications. For example, in some embodiments, Vip3A resistant H. zea are used in screening assays to test candidate pesticide agents (e.g., biopesticides).

For example, in some embodiments, candidate agents are contacted with Vip3A resistant H. zea described herein and one or more outcomes on viability (e.g., LC₅₀) or growth are assayed (e.g., using the method described in Example 1 or another suitable method).

Agents identified using the described compositions and methods find use in protecting a variety of crops against pests (e.g., pests that have evolved resistance to Bt crystalline (Cry) toxins). In some embodiments, the crops are agricultural crops or industrial crops.

In some embodiments, the Vip3Aa resistant H. zea described herein find use in research applications (e.g., determining fundamental understanding of Vip3a resistance), which improves design and screening of candidate products for controlling insect pests.

### EXAMPLES cl Example 1

This example describes selection of Vip3Aa resistant corn earworms.

TABLE 1
Strains of H. zea used in Vip3Aa selection.

| | | Selected with | Selected with |
|---|---|---|---|
| Strain | Origin | Vip3Aa | Cry1Ac |
| LAB-S | Long-term lab strain | No | No |
| GA | Georgia Bt corn field 2008 | No | Yes (field only) |
| GA-R | Subset of GA-R selected | No | Yes (field + lab) |
| | with Cry1Ac in lab | | |
| GZ | GA-R X LAB-S in 2018 | No | No |
| GZR3* | Subset of GZR selected | Yes | No |
| | with Vip3Aa in lab | | |

*To minimize negative effects of inbreeding, GZR3 was split into two subsets (GZR3A and GZR3B) that were crossed every second generation.

### Strains of Helicoverpa zea

Seven strains of H. zea were used (Table 1): LAB-S, GA, GA-R, GZ, GZR3, GZR3A, and GZR3B. LAB-S is a laboratory strain obtained from Benzon Research (Carlisle, Pa.) that has been reared without exposure to Bt toxins or other insecticides for many years. GA is a field-derived strain from Georgia that was exposed to Bt toxins only in the field. GA-R was derived from the GA strain and has been selected repeatedly in the laboratory for resistance to Cry1Ac (Welch et al. 2015). GZ was engineered with reciprocal mass crosses between GA-R and LAB-S, followed by rearing the progeny without exposure to Bt toxins. GZR3, GZR3A and GZR3B were derived from GZ and selected for resistance to Vip3Aa. To minimize negative effects of inbreeding, GZR3 was split into two subsets (GZR3A and GZR3B) that were crossed every second generation.

To generate the Vip3-resistant strain of Helicoverpa zea (aka corn earworm), larvae were reared on diet treated with Vip3Aa (a protein derived from Bacillus thuringiensis or Bt for short) and reared the survivors to continue the strain. This selection process was performed in each of more than 33 generations. Bioassays showed that the concentration of Vip3Aa killing 50% of larvae (LC₅₀) was 225-fold higher for the resistant strain (GZR3) relative to a Vip3Aa susceptible strain (GA) collected in Georgia and reared without exposure to Bt toxins in the laboratory. The LC50 of larvae from the first generation of progeny from a cross between GA and GZR3 was 18.8-fold higher than the LC50 for GA, showing that resistance to Vip3Aa in GZR3 was not completely recessive.

### Bt Toxin

Larvae were tested against Vip3Aa51 (Axmi005) provided by BASF. The amino acid homology is 94.9% between Vip3Aa19 used in Bt cotton and Vip3Aa51 (Sampson et al. 2008; US. Pat. No. 9,909,140). Vip3Aa51 protoxin tagged with a maltose-binding protein (MBP) was produced using recombinant E. coli. The tagged protein was purified on an amylose column. For simplicity, hereafter we refer to Vip3Aa51 as Vip3Aa.

### Rearing, Bioassays and Selection

GZR3A and GZR3B larvae were selected by exposing ca. 1000 to 2000 neonates from each strain to 1 to 10 μg Vip3Aa per cm² diet using the methods for rearing and diet overlay bioassays described by Welch et al. (J. Invert. Pathol. 132: 149-156 2015) and rearing the survivors to continue each strain.

The heterogeneous H. zea strain GZR3 was selected for more than 33 generations by exposing >1000 larvae each generation to Vip3Aa in diet. The data from March 2020 show the LC₅₀ of Vip3Aa for GZR3 was 18.8-fold higher than for the most susceptible strain (GA) and 16.4-fold higher than for its parent strain (GZ) (Table 2). The data from March 2021 show that the LC₅₀ for GZR3 was 225-fold higher than for GA. Furthermore in March 2021, the LC₅₀ of larvae from the first generation of progeny from a cross between GA and GZR3 was 18.8-fold higher than the LC₅₀ for GA, showing that resistance to Vip3Aa in GZR3 was not completely recessive.

TABLE 2
LC₅₀ of Vip3Aa for a selected strain and unselected strains of H. zea

| | | | | | |
|---|---|---|---|---|---|
| Strain | n | Slope (SE)^{a} | LC50^{b} | (95% FL) | RR^{c} |

Baseline (2018): before any selection with Vip3Aa

| | | | | | | |
|---|---|---|---|---|---|---|
| Benzon | 285 | 2.0 (0.3) | 1.71 | (0.82-3.0) | 7.9 | * |
| GZ^{d} | 238 | 1.6 (0.2) | 1 | (0.71-1.3) | 4.6 | * |
| GA-R | 571 | 2.1 (0.2) | 0.55 | (0.36-0.82) | 2.6 | * |
| GA | 558 | 2.7 (0.3) | 0.22 | (0.17-0.27) | 1 | |

GZ unselected, GZR3 selected with Vip3Aa

| | | | | | | |
|---|---|---|---|---|---|---|
| Nov. 2019 | | | | | | |
| GZ | 320 | 2.1 (0.2) | 0.43 | (0.32-0.56) | 2 | |
| GZR3 | 320 | 3.4 (0.8) | 2.64 | (2.0-3.9) | 12.2 | |
| Mar. 2020 | | | | | | |
| GZ | 320 | 2.0 (0.2) | 0.25 | (0.17-0.34) | 1.1 | |
| GZR3 | 320 | 1.9 (0.3) | 4.07 | (2.9-5.9) | 18.8 | * |
| Mar. 2021 | | | | | | |
| GA | 318 | 2.4 (0.3) | 0.31 | (0.05-0.64) | 1.0 | |
| GA X | 320 | 2.2 (0.2) | 5.82 | (4.0-8.5) | 18.8 | * |
| GZ3 | | | | | | |
| GZR3 | 319 | 2.6 (0.4) | 69.8 | (47-129) | 225 | * |

^{a}Slope of the concentration-mortality line with standard error in parentheses
^{b}Concentration killing 50% (μg toxin per cm² diet) with 95% fiducial limits in parentheses
^{c}Resistance ratio, LC50 for a strain divided by the LC50 for GA, the most susceptible strain
*LC50 significantly greater than LC50 for the most susceptible strain (GA), based on non-overlap of 95% FL
^{d}GZ obtained from reciprocal crosses between GA-R and Benzon and reared on untreated diet (unselected)
Note:
Based on survival to second instar after 1 week (mortality recorded as dead larvae + live first instars)

### Example 2

This example describes the use of genomic analyses to identify mutations associated with resistance to Vip3Aa.

To analyze the mechanism of resistance to Vip3Aa, genomic DNA sequences of five strains of H. zea: GZR3, GZ, GA-R, LAB-S and GA are compared. To reduce the noise from random genetic differences between strains and facilitate identification of genetic differences causally associated with resistance, GZR3 (Vip3Aa-resistant) and GZ (susceptible to Vip3Aa, parent strain of GZR3) are crossed to create GZR3 S, a heterogeneous strain that will harbor alleles for both resistance and susceptibility to Vip3Aa. To decrease linkage disequilibrium between alleles affecting responses to Vip3Aa and alleles at other loci that do not, GZR3S is reared without exposure to Bt toxins for 12 generations (ca. 1 year).

In ongoing work with H. zea, an established diet bioassay (Welch et al. J. Invert. Pathol. 132: 149-156 2015) is used to obtain resistant and susceptible larvae from GZR3S for genomic analyses. From GZR3 S, 4000 neonates are exposed to a low toxin concentration and 4000 neonates are exposed to a high toxin concentration. After 7 days, larvae exposed to the low concentration that are first instars are scored as susceptible, whereas those exposed to the high concentration that become third or later instars are scored as resistant. After scoring, all larvae are transferred to untreated diet and allowed to feed until they become fifth instars. This is expected to yield at least 200 susceptible and 200 resistant larvae, which are frozen at 80° C. for subsequent extraction of DNA for a genome-wide association study (GWAS).

Established methods are used for extracting and sequencing DNA from 192 susceptible and 192 resistant larvae from GZR3 S. Reads are mapped to an annotated genome of H. zea and analyzed to identify candidate genes for Vip3Aa resistance harboring mutations that occur at significantly higher frequency in resistant larvae than in susceptible larvae from GZR3 S. These mutations are filtered by also determining if they occur at significantly higher frequency in the Vip3Aa-resistant strain GZR3 vs. four strains susceptible to Vip3Aa: GZ, GA-R, GA, and LAB-S. This comprehensive approach enables identification of resistance genes not currently known to be involved in Bt toxicity or resistance, as well as testing of genes encoding proteins with reported roles in Bt toxicity, including four putative Vip3Aa receptors identified from Spodoptera frugiperda and Agrotis ipsilon, which are both lepidopteran pests in the same family as H. zea (Singh et al. Environ. Microbiol. 76: 7202-7209 2010, Chakroun et al. Microbiol. Mol. Biol. Rev. 80: 329-350 2016, Jiang et al. PLoS Pathogens 14(10): e1007347 2018, Jiang et al., Toxins 10: 546 2018).

All publications and patents mentioned in the above specification are herein incorporated by reference. Various modifications and variations of the described method and system of the disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure which are obvious to those skilled in the relevant fields are intended to be within the scope of the following claims.

1. A composition comprising a variant Helicoverpa zea, wherein said Helicoverpa zea is resistant to Vip3Aa.

2. The composition of claim 1, wherein said variant Helicoverpa zea exhibits an LC₅₀ of Vip3Aa at least 50 times higher than wildtype Helicoverpa zea.

3. The composition of claim 1, wherein said variant Helicoverpa zea exhibits an LC₅₀ of Vip3Aa at least 88 times higher wildtype Helicoverpa zea.

4. The composition of claim 1, wherein said variant Helicoverpa zea has one or more nucleic acid variations relative to wildtype Helicoverpa zea.

5. The composition of claim 4, wherein said variations are single nucleotide polymorphisms.

6. A kit or system, comprising:
the composition of claim 1.

7. The kit or system of claim 6, wherein said kit further comprises a Vip3A polypeptide.

8. The kit or system of claim 6, wherein said kit or system further comprises a candidate agent.

9. The kit or system of claim 8, wherein said candidate agent is a pesticide.

10. The kit or system of claim 9, wherein said pesticide is a biopesticide.

11. The kit or system of claim 10, wherein said biopesticide is a polypeptide.

12. The kit or system of claim 10, wherein said biopesticide is a bacterium.

13. A method of testing a candidate agent, comprising:
a) contacting the composition of claim 1 with said candidate agent; and
b) assaying the effect of said candidate compound on said Helicoverpa zea.

14. The method of claim 13, wherein said candidate agent is a pesticide.

15. The method of claim 14, wherein said pesticide is a biopesticide.

16. The method of claim 15, wherein said biopesticide is a polypeptide.

17. The method of claim 15, wherein said biopesticide is a bacterium.

18. The method of claim 13, wherein said effect is viability and/or growth.

19. The method of claim 18, wherein said effect is the LC₅₀ of said candidate agent.
